(19) United States
(12) Patent Application Publication
(10) Pub. No.: US 2025/0295833 A1
(43) Pub. Date: Sep. 25, 2025

(54) SCAFFOLDS FOR THE REGENERATION OF TISSUES AND PRODUCTION METHOD BY MEANS OF ELECTROSPINNING THEREOF

(71) Applicant: INSTITUTO DISTRITAL DE CIENCIA BIOTECNOLOGIA E INNOVACIÓN EN SALUD - IDCBIS, Bogotá (CO)

(72) Inventors: Leonardo PRIETO ABELLO, Bogotá (CO); Ingrid Zulay SILVA COTE, Bogotá (CO)

(21) Appl. No.: 18/864,284

(22) PCT Filed: May 12, 2023
(86) PCT No.: PCT/IB2023/054908

§ 371 Date: Nov. 8, 2024

(30) Foreign Application Priority Data
May 13, 2022 (CO) NC2022/0006262

(51) Int. Cl. A61L 27/18

(52) U.S. Cl. CPC A61L 27/18

(57) ABSTRACT

A scaffold comprising polycaprolactone (C₆H₁₀O₂)ₙ and glycine (C₂H₅NO₂), wherein the scaffold is prepared by electrospinning the polycaprolactone and glycine together; and the glycine has a gamma structural conformation. Such a scaffold is useful for the treatment of wounds, including wounds with an adverse environment, such as diabetic ulcers or pressure ulcers.

### FIELD OF THE INVENTION

The present invention relates to the field of medicine, more specifically to the field of biomedicine, even more specifically scaffolds for tissue regeneration and a procedure for obtaining scaffolds by electrospinning.

The invention has applicability in tissue engineering and regenerative therapy.

### STATE OF THE ART

Skin wounds such as pressure ulcers do not present a conducive environment for the cells surrounding these complex wounds to migrate and repair the tissue properly, which leads to incomplete or non-existent healing in many cases.

The use of biomaterials in Tissue Engineering has had an important impact in recent years from different combinations of materials and techniques, in order to provide cells with a support with structural integrity and supply of biological agents that favors a specific microenvironment of the tissue, regulates cellular functions and accelerates healing processes (Lizarazo-Fonseca, L., Prieto, E. M., Graziano, R. V., Camacho, B., Salguero, G., & Cote, I. S. (2019). Poly (e-caprolactone)/collagen electrospun scaffolds with potential use in skin tissue regeneration. Ciencia en Desarrollo, 10(2), 197-208.).

Different methods of processing biomaterials have been described, including hydrogels, 3D printing, electrospinning, among others. Electrospinning has been shown to be a promising technique in the field of skin regeneration due to the biomimicry of the fibers with the structure of the extracellular matrix.

Electrospinning is a technique that offers an excellent volume-to-area ratio, allows obtaining fibers with diameters ranging from submicron sizes to nanometric scales, is versatile and involves low processing costs.

For this technique a wide range of polymeric materials can be used, both natural and synthetic used to manufacture scaffolds by this technique, among them are: poly lactic acid (PLA), poly(glycolic acid) (PGA), poly(lactic-co-glycolic acid) (PLGA), poly (ϵ-caprolactone) (PCL) and natural polymers such as collagen and chitosan.

These polymers can be prepared, individually and in mixture of natural and synthetic polymers, in order to improve the properties of the scaffolds, taking into account the site of implantation (Lizarazo-Fonseca, L., et al. (2019)).

Specifically, the use of polycaprolactone for the generation of biosynthetic scaffolds by electrospinning is known in the art. However, polycaprolactone has excessive hydrophobicity, which hinders the attraction and growth of cells.

In this sense, Gautam et al. (S Gautam, A. K. Dinda, and N. C. Mishra, “Fabrication and characterization of PLC/gelatin composite nanofibrous scaffold for tissue engineering applications by electrospinning method” Material Sci. Eng. C., vol 33 N^{a}3, pp 1228-1235, 2013, doi: 10.1016/j.msec.2012.12.015) combined polycaprolactone with gelatin to make electrospun scaffolds and support fibroblast growth. They found that this combination was suitable to allow the growth of the aforementioned cells and, therefore, has great potential in the regeneration of tissues such as the skin. They used as solvents for the polymer solution a mixture of chloroform, methanol and acetic acid.

Dorati et al. (R. Dorati et al., “Study on hydrophilicity and degradability of chitosan/polylactide-co-polycaprolactone nanofiber blend electrospun membrane”, Carbohydrate Polymers, Volume 199, 1 Nov. 2018, Pages 150-160) made a solution with a copolymer of polycaprolactone and polylactic acid together with chitosan in a mixture of methylene chloride and dimethylformamide as solvents, which electrospun and obtained homogeneous scaffolds. They saw that mouse fibroblasts remained viable on these scaffolds for up to 3 days.

Bakhsheshi et al. (H. R. Bakhsheshi-Rad et al., “Antibacterial activity and in vivo wound healing evaluation of polycaprolactone-gelatin methacryloyl-cephatexin electrospun nanofibrous” Materials Letters, Volume 256, 1 Dec. 2019, 126618) successfully electrospun polycaprolactone with gelatin methacryloyl. The scaffolds obtained were loaded with cephalexin to give them antibacterial properties.

Zheng et al. (Zheng, W., Wang, Z., Song, L., Zhao, Q., Zhang, J., Li, D., & Kong, D. (2012). “Endothelialization and patency of RGD-functionalized vascular grafts in a rabbit carotid artery model.” Biomaterials, 33(10), 2880-2891) manufactured small diameter (2.2 mm) tubular grafts by electrospinning biodegradable polymeric polycaprolactone, followed by a functional surface coating with the RGD peptide.

Alazzawi et al. (Alazzawi, M., Kadim Abid Alsahib, N., & Turkoglu Sasmazel, H. (2021). “Core/Shell Glycine-Polyvinyl Alcohol/Polycaprolactone Nanofibrous Membrane Intended for Guided Bone Regeneration: Development and Characterization.” Coatings, 11(9), 1130) developed a nanofibrous membrane composed of polycaprolactone as a shell and polyvinyl alcohol:Gly as a core, using the coaxial electrospinning technique, characterizing it morphologically, mechanically, physically, chemically and thermally, intended for guided bone regeneration.

CN110507860A refers to a method for preparing blood vessels obtained by tissue engineering in situ and to the structure resulting from said method. In various of the steps of the method and layers of the generated structure, the use of the polycaprolactone and the electrospinning thereof are contemplated.

For its part, U.S. Ser. No. 10/245,353B2 discloses a hydrophilic electrospun biological scaffolding material that is prepared by mixing an aqueous solution of fibrinogen and L-arginine or hydrochloride thereof with a solution of Poly(L-lactide-co-epsilon-caprolactone) [P(LLA-CL)].

For its part, WO19144741A1 refers to a scaffold material including a charged fibrous skeleton material, which is covered with alternately superimposed positively charged biocompatible materials and negatively charged biocompatible materials by electrostatic attraction. The fibers are prepared by electrospinning and/or 3D printing, obtained by charge modification treatment, which constitute the material of the fibrous skeleton. It is contemplated that the fiber is a biocompatible polymeric material, which may be an organic synthetic polymeric material and/or a natural biopolymeric material. As a synthetic polymeric material, polycaprolactone is recited.

CN103520769A discloses a tissue engineered scaffolding material modified using growth factor (Mechano growth Factor, MgF) or its peptide E (MgF-Ct24E), composed of poly L-lactic acid (PLLA) or polycaprolactone (PCL) or a combination thereof, which promotes tissue repair, particularly the repair of bones, muscles, blood vessels and other tissues. The described PLLA, PCL or PLLA/PCL nanofiber scaffold is prepared by electrospinning technology, and crosslinking the MgF or MgF-Ct24E growth factor with the obtained nanofiber scaffold.

Therefore, there remains a need in the prior art for improved scaffolds, with improved physical properties (including hydrophilicity and fiber morphology) that allow the attraction or migration and proliferation of cells and that can be used for tissue regeneration, more preferably wound healing or repair, even in adverse situations, such as diabetic ulcers or pressure ulcers.

### DETAILED DESCRIPTION OF THE DRAWINGS

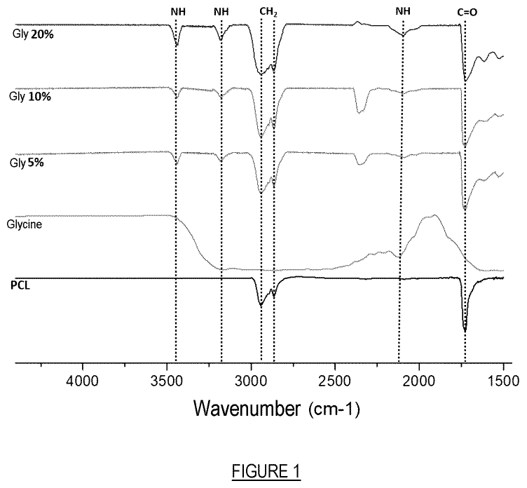

FIG. 1 shows the infrared spectroscopy (FTIR) spectrum obtained for four different scaffold formulations: one of polycaprolactone alone and three of polycaprolactone with different percentages by weight of glycine: 5%, 10% and 20%. Additionally, the spectrum for glycine powder alone was carried out.

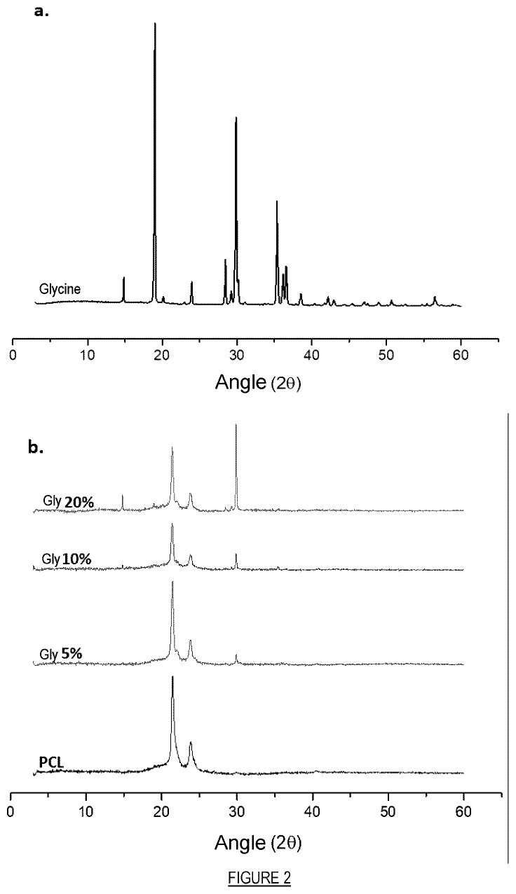

FIG. 2 shows the results of X-ray diffraction (XRD) of glycine alone (a) and of four different scaffold formulations (b): one of polycaprolactone alone and three of polycaprolactone with different percentages by weight of glycine: 5%, 10% and 20%.

FIG. 3 shows the results of contact angle (θ) at time 0 versus glycine content (%) obtained for four different scaffold formulations: one of polycaprolactone alone (square) and three of polycaprolactone with different weight percentages of glycine: 5% (circle), 10% (triangle), and 20% (inverted triangle).

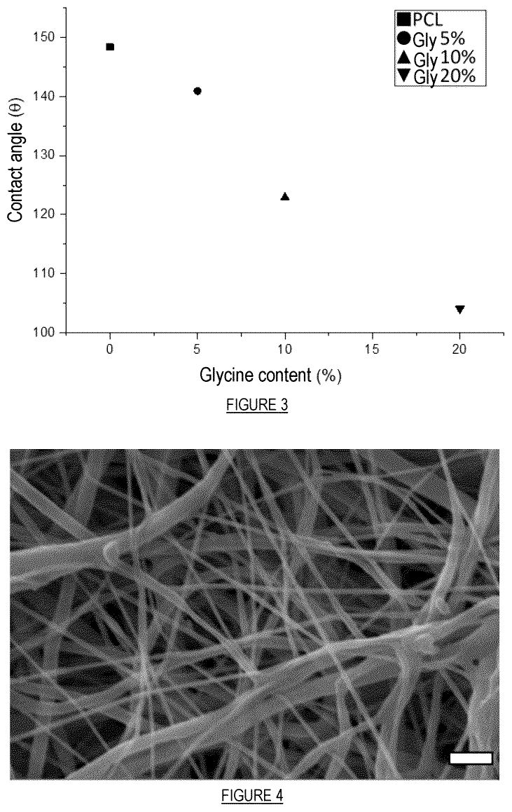

FIG. 4 corresponds to the results obtained by scanning electron microscopy (SEM) (5000×) for a polycaprolactone scaffold.

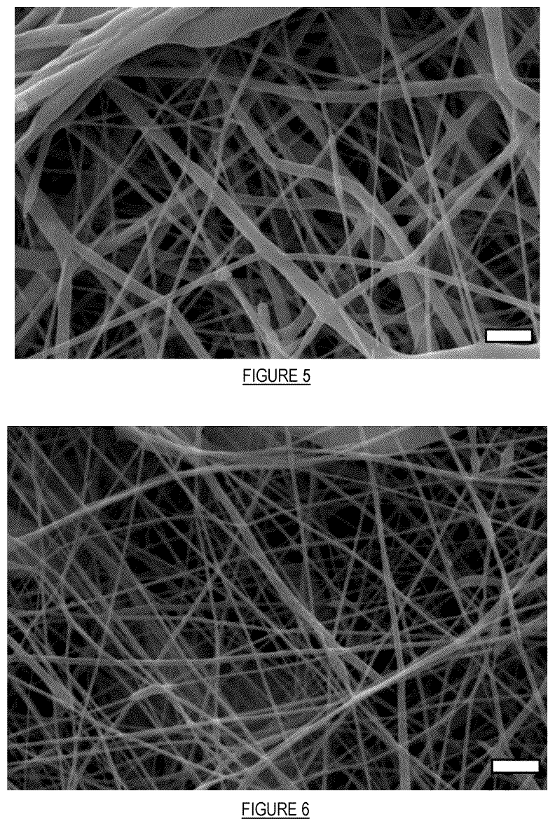

FIG. 5 corresponds to the results obtained by SEM (5000×) for a scaffold prepared with a solution of polycaprolactone and 5% glycine.

FIG. 6 corresponds to the results obtained by SEM (5000×) for a scaffold prepared with a solution of polycaprolactone and 10% glycine.

FIG. 7 corresponds to the results obtained by SEM (5000×) for a scaffold prepared with a solution of polycaprolactone and 20% glycine.

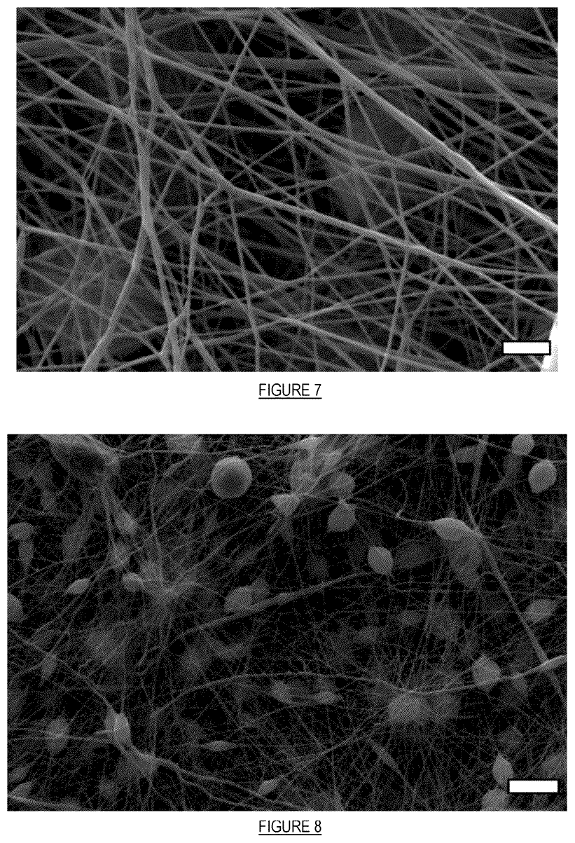

FIG. 8 corresponds to the results obtained by SEM (1000×) for a scaffold prepared with a solution of polycaprolactone and 10% glycine.

FIG. 9 corresponds to the results obtained by SEM (1000×) for a scaffold prepared with a solution of polycaprolactone and 20% glycine.

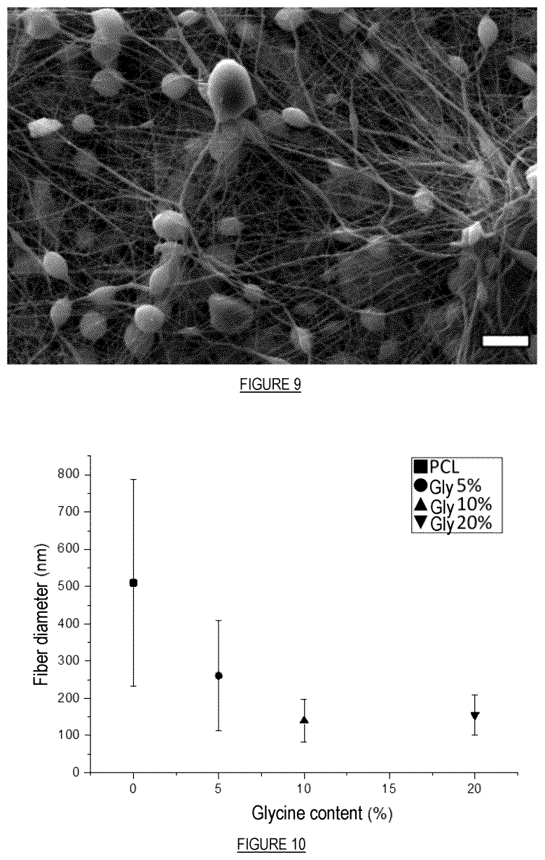

FIG. 10 shows the results obtained for four different scaffold formulations: one of polycaprolactone alone (square) and three of polycaprolactone with different percentages by weight of glycine: 5% (circle), 10% (triangle), and 20% (inverted triangle), relative to the average fiber diameter of the scaffolds as a function of the amount of glycine present in the solution used for electrospinning the scaffold.

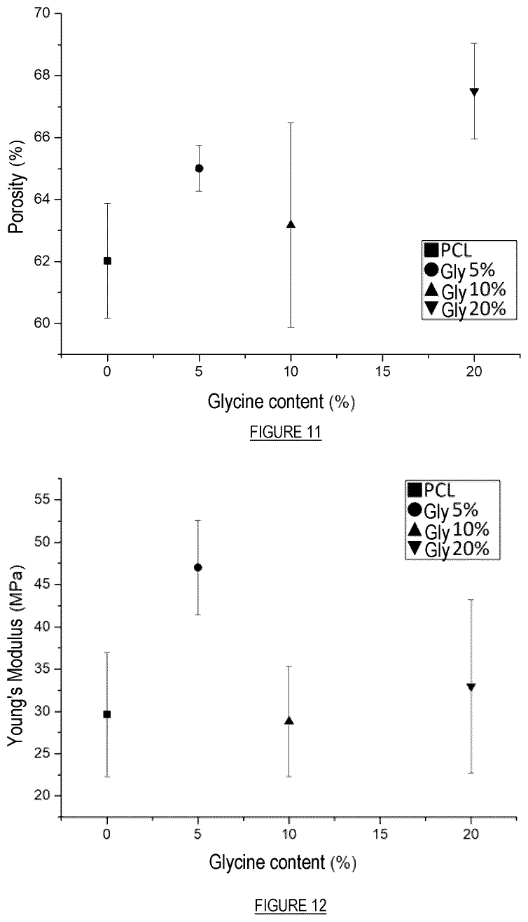

FIG. 11 shows the results obtained for four different scaffold formulations: one of polycaprolactone alone (square) and three of polycaprolactone with different percentages by weight of glycine: 5% (circle), 10% (triangle) and 20% (inverted triangle), regarding the porosity of the scaffolds as a function of the amount of glycine present in the solution used for electrospinning the scaffold.

FIG. 12 shows the results obtained for four different scaffold formulations: one of polycaprolactone alone (square) and three of polycaprolactone with different percentages by weight of glycine: 5% (circle), 10% (triangle), and 20% (inverted triangle), relative to the Young's Modulus of the scaffolds as a function of the amount of glycine present in the solution used for electrospinning the scaffold.

### DETAILED DESCRIPTION OF THE INVENTION

The present invention relates to a biosynthetic scaffold obtained by electrospinning polycaprolactone and glycine, which allow cell attraction and proliferation, which makes the cells surrounding the scaffold effectively help the repair of injuries and wounds, even in adverse situations, such as diabetic ulcers or pressure ulcers.

The incorporation of the glycine in the scaffold improves the hydrophilicity of the material and the morphology of the fibers making them thinner. In addition, since glycine is electrospun together with polycaprolactone, due to the parameters used in said electrospinning procedure, glycine changes its structural conformation from Alpha (α) to Gamma (γ), thus improving the adhesion and viability of different cell types.

Consequently, the biosynthetic scaffold of the present invention allows to solve the problems existing in the prior art and recited above.

In a first aspect, the present invention relates to a scaffold comprising polycaprolactone (C₆H₁₀O₂)ₙ and glycine (C₂H₅NO₂), characterized in that the scaffold is prepared by electrospinning together the polycaprolactone and the glycine, and the glycine has a gamma structural conformation.

The fact that the electrospinning of the polycaprolactone and the glycine is joint allows the glycine to be effectively included in the generated fibers. This inclusion of glycine in the electrospun polycaprolactone fibers allows to improve the hydrophilicity and morphology of said fibers (making them thinner and with morphology similar to that present in human skin), which contributes to the scaffold of the present invention generating an ideal environment so that once applied, the surrounding cells can migrate, proliferate and help in the repair of lesions, preferably wounds.

Additionally, as indicated above, the joint electrospinning allows the glycine to change its structural conformation from alpha to gamma (the change occurs due to the tension to which the glycine is subjected during the electrospinning process). This fact allows improving the adhesion and viability of various cell types.

Preferably, the fibers of the scaffold of the present invention are nanometric and/or submicrometric, more preferably nanometric and submicrometric. More preferably, the fibers have an average diameter of less than 300 nm; more preferably, the fibers have an average diameter of less than 300 nm and greater than 100 nm; more preferably, the fibers have an average diameter of between 275 nm and 100 nm; even more preferably, the fibers have an average diameter of between 160 and 140 nm.

During the electrospinning process, a combination of the amino groups of glycine generates a higher conductivity in the solution and the potential (voltage) difference applied during the process, causes the fibers to elongate and become thinner with respect to the common polycaprolactone fibers, this makes the scaffold have a morphology similar to that of the natural skin. In addition to the above, glycine improves cell adhesion, viability and proliferation in the scaffolds, an effect that is desirable in the repair or regeneration of skin.

In a preferred embodiment, the scaffold of the present invention has a porosity of at least 60%; more preferably between 60% and 90%; more preferably between 60% and 69%; even more preferably between 63% and 67%.

Also preferably, the Young's modulus of the scaffold of the present invention is at least 20 MPa, more preferably at least 25 MPa, more preferably at least 30 MPa, more preferably between 22.5 MPa and 52.5 MPa, even more preferably between 30 MPa and 47.5 MPa.

Preferably, the contact angle (θ) with water at time 0 seconds of the scaffold of the present invention is less than 150, more preferably less than 140, more preferably between 140 and 100, even more preferably between 120 and 100.

The scaffold of the present invention has a rough internal structure, preferably with beads that facilitate the adhesion and growth of the cells, more preferably the diameters of the beads are between 3 and 8 μm. This internal structure is given by the effect of the incorporation of glycine in the manner explained above.

In a second aspect, the present invention relates to a procedure for obtaining the scaffold of the present invention by electrospinning, comprising the following steps:
a) Preparation of the polycaprolactone and glycine polymeric solution.
b) Electrospinning of the polymeric solution obtained in step a) to obtain the scaffold of the present invention.

In step a), preferably, a solution is prepared with polycaprolactone and glycine in an 8:2 ratio, more preferably at a concentration of at least 10% w/v (weight/volume) in an organic solvent, more preferably between 10% w/v and 20% w/v, even more preferably 10% w/v. The organic solvent is preferably trifluoroethanol for synthesis (C₂H₃F₃O).

After preparation of the polymeric solution said solution is preferably left under stirring, even more preferably at least 10 hours at 550-800 rpm and 50-65° C.

In step b), preferably, the polymeric solution obtained in step a) is introduced into a syringe with a stainless steel needle. This syringe is located in a syringe injection pump which will allow controlled dosing of the polymeric solution during electrospinning. An aluminum-coated copper plate is located 15 cm from the tip of the needle. The fibers will be deposited on this plate during electrospinning. In addition, a voltage source (preferably with the capacity to provide electrical voltage of up to 30 kV) is connected to the needle and the collector plate.

In a preferred embodiment, in step b), the polymeric solution obtained in step a) is injected at a rate of 1 to 1.5 ml/h, preferably 1.3 ml/h, and between 15-19 kV of electrical voltage, preferably 17 kV, is applied (to volatilize the solvent). The scaffold obtained has a white color.

Preferably, in step b), after electrospinning, the scaffold obtained must be allowed to dry at least 7 days at 37° C. to obtain the scaffold of the present invention.

It is contemplated that the scaffold of the present invention is preferably in the form of a porous membrane. This porous membrane would be suitable for cell infiltration and would have mechanical properties suitable for the final application of the product on, for example, human skin.

In a preferred embodiment the wound is a skin wound, more preferably an acute or chronic skin wound, even more preferably the wound is a diabetic ulcer or a pressure ulcer.

In a final aspect the present invention relates to a method for tissue regeneration characterized in that it comprises the administration of a scaffold of the present invention, to a patient in need of the treatment.

The scaffold of the present invention is as explained above in the second embodiment of the present invention.

More preferably, in this final aspect of the present invention, the invention relates to a method for the treatment of a wound characterized in that it comprises the administration of a scaffold of the present invention, to a patient in need of the treatment.

In a preferred embodiment the wound is a skin wound, more preferably an acute or chronic skin wound, even more preferably the wound is a diabetic ulcer or a pressure ulcer.

The patient in need of treatment is preferably a mammal, more preferably a human.

### EXAMPLES

### Example 1. Procedure for Obtaining a Scaffold by Electrospinning According to the Present Invention

In the present example 4 different scaffold formulations were electrospun, one of polycaprolactone alone and 3 of polycaprolactone with different weight percentages of glycine: 5%, 10% and 20%. Additionally, a sample of glycine powder alone was used.

The electrospinning parameters used for the manufacture of these scaffolds were 17 kV, needle tip to manifold distance of 15 cm, and injection rate of 1.3 mL/h. Below are the results obtained for the scaffolds obtained.

### Results by Infrared Spectroscopy (FTIR)

To verify that polycaprolactone and glycine were present in the fibers and, therefore, were properly integrated into the scaffold, infrared spectroscopy (FTIR) was performed using the attenuated total reflection (ATR) technique for the scaffolds and the KBr pellet technique for glycine. The spectra of the glycine powder and the scaffolds made of polycaprolactone, and the mixtures of polycaprolactone with 5%, 10% and 20% of glycine are shown in FIG. 1.

In the spectra obtained by FTIR, the signal of 3180 cm⁻¹ corresponds to the asymmetric stretching of NH and the signal of 2100 cm⁻¹ corresponds to turns of the NH₃ bond in the spectrum of α-glycine (T. P. Srinivasan, R. Indirajith, and R. Gopalakrishnan, “Growth and characterization of α and γ-glycine single crystals,” J. Cryst. Growth, vol. 318, no. 1, pp. 762-767, 2011, doi: 10.1016/j.jcrysgro.2010.11.117) one of the crystalline forms of this amino acid. In the spectrum of polycaprolactone alone, the peaks at 2945, 2865 and 1725 cm⁻¹ stand out, the first two correspond to vibrations of CH₂ bonds and the third to vibrations of C═O bonds (F. Rojo-Callejas, “Infrared Spectroscopy Tables,” Fac. Chemistry—Dep. Audiovisual Programs—Univ. San Carlos Guatemala, p. 11, 2000, [online]. Available on Apr. 22, 2022 at: http://depa.fquim.unam.mx/amyd/archivero/TablasIR_15437.pdf%0Ahttp://depa.fquim.u nam.mx/amyd/archivero/IRTablas_33080.pdf). The five peaks recited above are present in the three spectra obtained from the scaffolds of the present invention obtained with the mixture of polycaprolactone and glycine. In said scaffolds of the present invention, in addition, the appearance of a new peak at wave number 3436 cm⁻¹ is observed, which is related to the symmetrical stretching of the NH bonds, this is characteristic of γ-glycine (E. Ramachandran, K. Baskaran, and S. Natarajan, “XRD, thermal, FTIR and SEM studies on gelgrown γ-glycine crystals,” Cryst. Res. Technol., vol. 42, no. 1, pp. 73-77, 2007, doi: 10.1002/crat.200610774), another crystalline form of glycine.

The different peaks obtained in FTIR are described in the following Table 1.

TABLE 1
FTIR spectral data of polycaprolactone, glycine, and
scaffolds made of polycaprolactone and glycine.

| Wavenumber (cm⁻¹) | Description |
|---|---|
| 3436 | NH symmetrical stretching |
| 3180 | NH asymmetrical stretching |
| 2945 | CH₂ vibration |
| 2865 | CH₂ vibration |
| 2100 | NH Torque |
| 1725 | C═O vibration |

The foregoing indicates that with the electrospinning process the crystal structure of glycine changed from α-glycine to γ-glycine.

### X-Ray Diffraction (DRX) Results

To study the crystal structure of the electrospun scaffolds, an XRD analysis was performed. Polycaprolactone is a semi-crystalline polymer and glycine is a crystalline solid with three different structural conformations (M. Rabesiaka, et al., “Preparation of glycine polymorphs crystallized in water and physicochemical characterizations,” J. Cryst. Growth, vol. 312, no. 11, pp. 1860-1865, 2010, doi: 10.1016/j.jcrysgro.2010.03.011) so it is important to analyze not only the structure but the contribution of both materials in the total crystallinity of the scaffolds.

The diffractogram of glycine (FIG. 2A) showed a highly crystalline structure corresponding to the α-glycine conformation (M. Matsumoto, Y. Wada, and K. Onoe, “Change in glycine polymorphs induced by minute-bubble injection during antisolvent crystallisation,” Adv. Powder Technol., vol. 26, no. 2, pp. 415-421, 2015, doi: 10.1016/j.apt.2014.11.014). In FIG. 2B, the diffractogram of the polycaprolactone showed an amorphous region and two crystalline peaks because it is a semi-crystalline solid, this characteristic was repeated in the diffractograms of the electrospun polycaprolactone and the scaffolds made with the mixtures of polycaprolactone and glycine. In the latter case, mixtures with 5% and 10% glycine showed an additional peak to those of polycaprolactone at angle 2θ=30, and the mixture with 20% glycine showed an additional peak formed near angle 2θ=15.

The intensity of these peaks was greater as the amount of glycine was increased, indicating that glycine not only integrated with polycaprolactone, but did so in a crystal structure that corresponds to the conformation of γ-glycine crystals (M. Anis, et al., “Novel report on γ-glycine crystal yielding high second harmonic generation efficiency,” Opt. Mater. (Amst)., vol. 72, pp. 590-595, 2017, doi: 10.1016/j.optmat.2017.07.007). This confirmed that the crystal structure of glycine changed from α-glycine to γ-glycine, supporting the results obtained with the FTIR and shown above.

### Contact Angle Results

The evaluation of the hydrophilicity of the scaffolds was made by measuring the contact angle between the scaffolds and a drop of water. To see the effect that glycine has on the scaffolds, the contact time of 0 seconds was considered, since at that time it is possible to directly analyze how much the glycine content influences this property. A lower contact angle indicates a better interaction between the material and the water and therefore better wettability.

The results obtained are summarized in FIG. 3.

It was observed that the contact angle decreased as the glycine content increased, which was due to the NH₂ and OH groups present in the glycine, which made the polycaprolactone and glycine scaffolds more hydrophilic. The best performing scaffold in the contact angle test was the one that had 20% glycine in the formulation used for electrospinning the scaffold.

### Scanning Electron Microscopy (SEM) Results

One of the main characteristics necessary for the application of scaffolds for wound healing is to achieve a fiber diameter less than 300 nm and a porosity greater than 60% to achieve the structure most similar to the extracellular matrix present in the skin (Z. Ma, M. Kotaki, and R. Inai, “Potential of Nanofiber Matrix as Tissue-Engineering Scaffolds,” Tissue Eng., vol. 11, no. 1, pp. 101-109, 2005). SEM images were used to study the morphology of the manufactured scaffolds.

The images obtained by SEM are shown in FIGS. 4 to 7. Fibers with micron diameter and random orientation appear clearly in the images. It was observed that the diameter of the fibers decreased as the glycine content in the scaffold increased, with the scaffolds with 10% and 20% glycine being the smallest in diameter with no significant difference between them. Additionally, and surprisingly, it was possible to observe that in the manufactured scaffolds using 10 or 20% glycine, beads were formed (see FIGS. 8 and 9), mainly because when the concentration of polymer in the solution was reduced, smaller diameters were caused in the fibers and the formation of these internal structures of the bead-like scaffold.

FIG. 10 shows the decrease in fiber diameter as the glycine content increases. The diameter of the fibers is strongly related to the conductivity of the solution, and the conductivity could be increased due to the amino groups provided by glycine (S. Ramakrishna, et al., “An introduction to electrospinning and nanofibers,” An Introd. to Electrospinning Nanofibers, pp. 1-382, January 2005, doi: 10.1142/5894/SUPPL_FILE/5894_CHAP1.PDF). The higher conductivity caused a greater stretching of the fibers in their path from the needle tip to the collector during the electrospinning process and, therefore, caused smaller fiber diameters in the scaffold (Y. M. Ju, et al., “Bilayered scaffold for engineering cellularized blood vessels,” 2010, doi: 10.1016/j.biomaterials.2010.02.002).

As for porosity, the ideal for the application of scaffolds in the regeneration of tissues and, more preferably, of wounds, is between 60% and 90% to improve cellular infiltration (Z. Yang, et al., “Crystallization behavior of poly(e-caprolactone)/layered double hydroxide nanocomposites,” J. Appl. Polym. Sci., vol. 116, no. 5, pp. 2658-2667, 2010, doi: 10.1002/app). It can be appreciated from FIG. 10 that as the glycine content increased, the porosity of the scaffolds tended to increase. However, all the scaffolds evaluated had a porosity greater than 60% and, therefore, were within the range to allow adequate cellular infiltration.

### Young's Modulus Results

The evaluation of Young's modulus is essential to verify if the scaffolds can withstand the stresses to which the skin is subjected. For this reason, this property was measured in the different manufactured scaffolds. The results obtained are summarized in FIG. 11. As can be seen in said Figure, it was possible to see that with the lowest glycine content evaluated, which is 5%, the Young's modulus was greater than that presented by the scaffold made only with the polymer, as if the glycine in said situation acted as a reinforcement of the polycaprolactone structure, while when the glycine content increased to 10% and 20% this property returned to have values similar or lower than those obtained by the polymer alone.

It is important to note, as already indicated above, that scaffolds prepared with formulations with 10% or 20% glycine are the only ones that have beads in their morphology (see FIG. 8). This morphological characteristic was able to decrease the mechanical properties of the material (A. Barhoum, M. Bechelany, and A. Hamdy, Handbook of Nanofibers. 2019).

However, it is interesting that, despite the reduction of the diameter of the fibers and the presence of said beads as an internal structure with the addition of glycine, at least mechanical properties similar to those of the polymer alone were maintained, validating the use of the scaffolds of the present invention for application to skin for wound regeneration. In this regard, it should be noted that in the prior art the Young's modulus of electrospun scaffolds made of polycaprolactone and gelatin has been reported, said Young's modulus being 3 MPa and it is stated that this value would be sufficient for a material used in tissue repair (Z. Wang et al., “Fabrication and in vitro evaluation of PCL/gelatin hierarchical scaffolds based on melt electrospinning writing and solution electrospinning for bone regeneration,” Mater. Sci. Eng. C, vol. 128, no. May, p. 112287, 2021, doi: 10.1016/j.msec.2021.112287). The scaffolds of the present invention reached a much higher Young's modulus and, therefore, this confirms that in relation to this property they could be used in tissue repair applications and, more preferably, of the skin.

### Cell Viability Results

To study the viability of mesenchymal stromal cells (MSCs) in the scaffolds of the present invention, the live/dead reagent was used to determine whether or not such scaffolds have a cytotoxic effect and are therefore suitable for use as a scaffold for tissue repair (and more preferably of skin wounds). All cells under the different conditions tested were colored green (living cells) and only a few were colored red (dead cells). This could be seen in three times (24 h, 72 h, 120 h) for the four scaffolds and the control. This indicates that all scaffolds manufactured provided conditions to maintain viable cells.

### CONCLUSIONS

Therefore, it could be concluded that glycine, in the compositions and scaffolds of the present invention, changed its α-to-γ structural conformation, which provided good mechanical properties, a morphological change in the fibers making them more similar to the natural fibers of the dermis, improved the hydrophilicity and viability of mesenchymal stromal cells in the scaffold.

The scaffolds with the highest contents of glycine (10% and 20% in the formulation used for the preparation of the scaffold of the present invention) were the most suitable for use in skin repair because they were the ones that came closest to the morphology of the fibers present in the natural dermis, internal structures in the form of a bead were observed that allow better cell adhesion, they presented good mechanical properties, good hydrophilicity and adequate cell viability.

1. A scaffold comprising polycaprolactone (C6H10O2)n and glycine (C2H5NO2), characterized in that the scaffold is prepared by electrospinning together the polycaprolactone and the glycine, wherein the glycine has a gamma structural conformation.

2. The scaffold of claim 1, wherein the glycine is included within fibers of the polycaprolactone.

3. The scaffold of claim 2, wherein the fibers of the polycaprolactone are nanometric and/or submicrometric in size.

4. The scaffold of claim 1, wherein said scaffold has a rough internal structure.

5. The scaffold of claim 4, wherein the rough internal structure is beads.

6. The scaffold of claim 1, wherein the scaffold is in the form of a porous membrane.

7. A procedure of electrospinning the scaffold according to claim 1, comprising the following steps:
a) preparing the polycaprolactone and glycine polymeric solution; and
b) electrospinning the polymeric solution obtained in step a) to obtain the scaffold.

8. The procedure according to claim 7, wherein in step a), the polycaprolactone and the glycine are in an 8:2 ratio.

9. The procedure according to claim 7, wherein the polycaprolactone and the glycine are at a concentration of at least 10% w/v in an organic solvent.

10. The procedure according to claim 9, wherein the polycaprolactone and glycine are at a concentration of between 10% w/v and 20% w/v.

11. The procedure according to claim 9, wherein the organic solvent is trifluoroethanol.

12. The procedure according to claim 7, wherein after the preparation of the polymeric solution in step a) said solution is left at least 10 hours at 550-800 rpm and 50-65° C.

13. The procedure according to claim 7, wherein in step b), the polymeric solution obtained in step a) is injected for electrospinning at a rate of 1 to 1.5 ml/h and 15-19 kV of electric voltage is applied.

14. The procedure according to claim 7, wherein, in step b), after electrospinning, the scaffold obtained is allowed to dry for at least 7 days at 37° C.
